(19) United States
(12) Patent Application Publication
(10) Pub. No.: US 2025/0281664 A1
(43) Pub. Date: Sep. 11, 2025

(54) BIOCOMPATIBLE ADHESIVE MATERIALS AND METHODS OF USE

(71) Applicant: BioDevek, Inc., Cambridge, MA (US)

(72) Inventors: Gonzalo Munoz Taboada, Cambridge, MA (US); Elazer R. Edelman, Brookline, MA (US); Natalie Artzi, Brookline, MA (US)

(21) Appl. No.: 18/860,080

(22) PCT Filed: Apr. 28, 2023
(86) PCT No.: PCT/US2023/020393

§ 371 Date: Oct. 25, 2024

Related U.S. Application Data
(60) Provisional application No. 63/336,528, filed on Apr. 29, 2022.

(51) Int. Cl. A61L 24/04; A61L 24/00

(52) U.S. Cl. CPC A61L 24/046; A61L 24/0015

(57) ABSTRACT

Biocompatible adhesive materials, such as for use with biological tissues and/or medical implants, are provided, as well as methods and kits for making and using the biocompatible adhesive materials. The biocompatible adhesive materials include a functionalized poloxamer component and an oxidized polysaccharide component, and may be tailored for specific tissue types and conditions.

### RELATED APPLICATIONS

This application claims the benefit of priority to U.S. Provisional Patent Application No. 63/336,528, filed Apr. 29, 2022.

### BACKGROUND

A number of tissue adhesives have been used in various medical procedures and applications, including topical wound closure, supplementing or replacing surgical sutures or staples, adhesion of synthetic materials to biological tissues, and drug delivery. A number of known tissue adhesives, however, are unsuitable for many applications, for example, due to toxic degradation products, slow curing, poor mechanical strength, and other drawbacks.

Several varieties of hydrogel adhesives have been developed, which are nontoxic and have improved properties. These hydrogels are generally formed by reacting a component having nucleophilic groups with a component having electrophilic groups that react to form a crosslinked network. However, these hydrogels typically dissolve too quickly, lack sufficient adhesion, or have insufficient mechanical strength.

Therefore, it would be desirable to provide improved adhesive formulations that overcome one or more of the above-described disadvantages.

### SUMMARY

Disclosed are biocompatible adhesive materials designed to seal, coat, bond, or protect biological and prosthetic materials.

In an aspect, disclosed is a biocompatible adhesive material, comprising:
a block copolymer comprising one or more polyethylene oxide (PEO) blocks, one or more polypropylene oxide (PPO) blocks, and at least one primary amine group; and
an oxidized polysaccharide comprising at least two aldehyde moieties;
wherein the adhesive material is crosslinked by a plurality of imine moieties formed from the at least one primary amine of the block copolymer and the at least two aldehyde moieties of the oxidized polysaccharide; and the adhesive material is a hydrogel.

In an aspect, disclosed is a biocompatible adhesive material, comprising:
a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine; and
an oxidized polysaccharide comprising at least two aldehyde moieties;
wherein the adhesive material is crosslinked by a plurality of imine moieties formed from the at least one primary amine of the linker-amine moiety of the functionalized poloxamer and an aldehyde moiety of the oxidized polysaccharide; and the adhesive material is a hydrogel.

In an aspect, disclosed is a method for treating, adhering, or sealing one or more biological tissues or prosthetic materials, the method comprising:
providing a block copolymer comprising one or more polyethylene oxide (PEO) blocks, one or more polypropylene oxide (PPO) blocks, and at least one primary amine group;
providing a second component comprising an oxidized polysaccharide comprising at least two aldehyde moieties;
combining the first component and the second component, thereby producing an adhesive formulation;
contacting the adhesive formulation with a surface of one or more biological tissues; and
allowing the adhesive formulation to cure in contact with the surface of the one or more biological tissues.

In an aspect, disclosed is a method for treating, adhering, or sealing one or more biological tissues or prosthetic materials, the method comprising:
providing a first component comprising a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine;
providing a second component comprising an oxidized polysaccharide comprising at least two aldehyde moieties;
combining the first component and the second component, thereby producing an adhesive formulation;
contacting the adhesive formulation with a surface of one or more biological tissues; and
allowing the adhesive formulation to cure in contact with the surface of the one or more biological tissues.

In an aspect, disclosed is a kit, comprising:
a first component, comprising a block copolymer comprising one or more polyethylene oxide (PEO) blocks, one or more polypropylene oxide (PPO) blocks, and at least one primary amine group; and a second component, comprising an oxidized polysaccharide comprising at least two aldehyde moieties;
a first syringe; and
instructions for use.

In an aspect, disclosed is a kit, comprising:
a first component, comprising a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine; and a second component, comprising an oxidized polysaccharide comprising at least two aldehyde moieties;
a first syringe; and
instructions for use.

In an aspect, disclosed is a drug delivery composition, comprising:
a biocompatible adhesive material disclosed herein; and
an effective amount of at least one drug;
wherein the at least one drug is admixed with the biocompatible adhesive material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 is a scheme showing a strategy to build the family of modified poloxamers.

FIG. 2 shows mechanisms of gelation of the hydrogel. Amine-modified poloxamers can achieve thermal gelation; the addition of oxidized dextran can achieve a quick gelation independent of temperature.

FIG. 3 shows a scheme of covalent bond interactions.

FIG. 4 shows a scheme of ionic interactions.

FIG. 5 shows a scheme of mechanical interlocking.

FIG. 6 shows a surface response model of the gelation time when using different concentrations of oxidized dextran and PEI modified poloxamer-188 (PluPEI).

FIG. 7 shows a surface response model of the hydrogel swelling when combining different concentrations of PEI modified poloxamer 188 (PluPEI) and oxidized dextran.

FIG. 8A shows that a thin layer of the material can be bent up to 180 degrees and the material does not develop cracks or breaks.

FIG. 8B shows that a thick layer of the material can be bent up to 180 degrees and the material does not develop cracks or breaks.

FIG. 9 shows the burst pressure of several dextran-PluPEI hydrogels at to and t24.

FIG. 10 shows the buffering capacity displayed by different hydrogel formulations.

FIG. 11 shows the swelling of specific formulations at 24 h.

FIG. 12 shows the in-vitro drug release profile of acriflavine of several hydrogel formulations.

### DETAILED DESCRIPTION

Improved biocompatible adhesive materials, compositions, and methods have been developed for adhering, sealing, or treating one or more biological tissues. Generally, these biocompatible adhesive materials comprise a functionalized poloxamer component and an oxidized polysaccharide component. In some embodiments, the adhesive formulations are used as tissue adhesives, tissue sealants, tissue treatments, matrix materials, fillers, coatings, or a combination thereof.

In an aspect, disclosed is a biocompatible adhesive material, comprising:
a block copolymer comprising one or more polyethylene oxide (PEO) blocks, one or more polypropylene oxide (PPO) blocks, and at least one primary amine group; and
an oxidized polysaccharide comprising at least two aldehyde moieties;
wherein the adhesive material is crosslinked by a plurality of imine moieties formed from the at least one primary amine of the block copolymer and the at least two aldehyde moieties of the oxidized polysaccharide; and the adhesive material is a hydrogel.

In certain embodiments, the block copolymer has a molecular weight of about 1,000 Da to about 25,000 Da, or about 1,000 Da to about 20,000 Da.

In certain embodiments, the block copolymer is inverse thermosensitive.

In certain embodiments, the block copolymer comprises 1 to about 50 primary amines. In certain embodiments, the block copolymer comprises 1 to about 25 primary amines.

In certain embodiments, the block copolymer has a PEO blocks: PPO blocks ratio of about 50:50 to about 80:20. The PEO blocks: PPO blocks ratio is the ratio of the number of PEO units to the number of PPO units.

In certain embodiments, the block copolymer is FG-(poloxamer 188)-FG, FG-(poloxamer 237)-FG, FG-(poloxamer 338)-FG, FG-(poloxamer 407)-FG, or a combination of any of them, wherein FG independently for each occurrence represents H or a primary amine group, at least one occurrence of FG is not H.

In certain embodiments, independently for each occurrence the primary amine group is 1,2-diaminoethane, tris (2-aminoethyl) amine, or a low molecular weight (e.g., less than about 2,000, 1,000, 500, or 250 Daltons) polyethyleneimine. For example, the primary amine group is a 1, 2-diaminoethane residue, a tris (2-aminoethyl) amine residue, or a combination thereof. In certain embodiments, the primary amine group is provided by a modification of a poloxamer with 1,2-diaminoethane, tris (2-aminoethyl) amine, or a combination thereof.

In an aspect, disclosed is a biocompatible adhesive material, comprising:
a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine; and
   an oxidized polysaccharide comprising at least two aldehyde moieties;
wherein the adhesive material is crosslinked by a plurality of imine moieties formed from the at least one primary amine of the linker-amine moiety of the functionalized poloxamer and an aldehyde moiety of the oxidized polysaccharide; and the adhesive material is a hydrogel.

In certain embodiments, the both occurrences of FG represent a linker-amine moiety.

In certain embodiments, the functionalized poloxamer has a molecular weight of about 1,000 Da to about 25,000 Da, or about 1,000 Da to about 20,000 Da.

In certain embodiments, the functionalized poloxamer is inverse thermosensitive.

In certain embodiments, each linker-amine moiety comprises 1 to about 50 primary amines.

In certain embodiments, each linker-amine moiety comprises 1 to about 25 primary amines.

In certain embodiments, the functionalized poloxamer has a PEO: PPO ratio of about 50:50 to about 80:20. The PEO: PPO ratio is the ratio of the number of PEO units to the number of PPO units.

In certain embodiments, the functionalized poloxamer is FG-(poloxamer 188)-FG, FG-(poloxamer 237)-FG, FG-(poloxamer 338)-FG, FG-(poloxamer 407)-FG, or a combination of any of them.

In certain embodiments, independently for each occurrence the amine moiety of the linker-amine moiety is 1,2-diaminoethane, tris (2-aminoethyl) amine, or a low molecular weight (e.g., less than about 2,000, 1,000, 500, or 250 Daltons) polyethyleneimine. For example, the amine moiety of the linker-amine moiety is a 1, 2-diaminoethane residue, a tris (2-aminoethyl) amine residue, or a combination thereof. In certain embodiments, the amine moiety of the linker-amine moiety is provided by a modification of a poloxamer with 1,2-diaminoethane, tris (2-aminoethyl) amine, or a combination thereof.

In certain embodiments, the oxidized polysaccharide has a molecular weight of about 1,000 Da to about 100,000 Da.

In certain embodiments, the degree of functionalization of the oxidized polysaccharide is about 10% to about 75%.

In certain embodiments, the oxidized polysaccharide comprises 2 to 100 aldehyde moieties.

In certain embodiments, the oxidized polysaccharide comprises 40 to 80 aldehyde moieties.

In certain embodiments, the oxidized polysaccharide comprises 50 to 70 aldehyde moieties.

In certain embodiments, the oxidized polysaccharide is a dextran.

In certain embodiments, the dextran has a molecular weight of about 10 kDa.

In certain embodiments, about 50% of the hydroxyl groups of the dextran are oxidized to aldehydes.

In certain embodiments, the adhesive material has a flexibility to bend up to 180 degrees. In certain embodiments, a three centimeter long sample of the adhesive material has a flexibility to bend up to 180 degrees.

In an aspect, disclosed is a method for treating, adhering, or sealing one or more biological tissues or prosthetic materials, the method comprising:
providing a block copolymer comprising one or more polyethylene oxide (PEO) blocks, one or more polypropylene oxide (PPO) blocks, and at least one primary amine group;
providing a second component comprising an oxidized polysaccharide comprising at least two aldehyde moieties;
combining the first component and the second component, thereby producing an adhesive formulation;
contacting the adhesive formulation with a surface of one or more biological tissues; and
allowing the adhesive formulation to cure in contact with the surface of the one or more biological tissues.

In certain embodiments, the method further includes the step of adjusting the concentrations of the first component or the second component to compensate for the characteristics of the biological tissue.

In certain embodiments, the block copolymer of the method has a molecular weight of about 1,000 Da to about 25,000 Da, or about 1,000 to about 20,000 Da.

In certain embodiments, the block copolymer of the method is inverse thermosensitive.

In certain embodiments, the block copolymer of the method comprises 1 to about 50 primary amines.

In certain embodiments, the block copolymer of the method comprises 1 to about 25 primary amines.

In certain embodiments, the block copolymer of the method has a PEO: PPO ratio of about 50:50 to about 80:20.

In certain embodiments, the block copolymer of the method is FG-(poloxamer 188)-FG, FG-(poloxamer 237)-FG, FG-(poloxamer 338)-FG, FG-(poloxamer 407)-FG, or a combination of any of them, wherein FG independently for each occurrence represents H or a primary amine group, at least one occurrence of FG is not H.

In certain embodiments, independently for each occurrence the primary amine group of the method is 1,2-diaminoethane, tris (2-aminoethyl) amine, or a low molecular weight (e.g., less than about 2,000, 1,000, 500, or 250 Daltons) polyethyleneimine. For example, the primary amine group is a 1, 2-diaminoethane residue, a tris (2-aminoethyl) amine residue, or a combination thereof. In certain embodiments, the primary amine group is provided by a modification of a polaxomer with 1,2-diaminoethane, tris (2-aminoethyl) amine, or a combination thereof.

In an aspect, disclosed is a method for treating, adhering, or sealing one or more biological tissues or prosthetic materials, the method comprising:
providing a first component comprising a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine;
providing a second component comprising an oxidized polysaccharide comprising at least two aldehyde moieties;
combining the first component and the second component, thereby producing an adhesive formulation;
contacting the adhesive formulation with a surface of one or more biological tissues; and
allowing the adhesive formulation to cure in contact with the surface of the one or more biological tissues.

In certain embodiments, the method further includes the step of adjusting the concentrations of the first component or the second component to compensate for the characteristics of the biological tissue.

In certain embodiments, the functionalized poloxamer of the method has a molecular weight of about 1,000 Da to about 25,000 Da, or about 1,000 to about 20,000 Da.

In certain embodiments, the functionalized poloxamer of the method is inverse thermosensitive.

In certain embodiments, each linker-amine moiety of the method comprises 1 to about 50 primary amines.

In certain embodiments, each linker-amine moiety of the method comprises 1 to about 25 primary amines.

In certain embodiments, the functionalized poloxamer of the method has a PEO: PPO ratio of about 50:50 to about 80:20.

In certain embodiments, the functionalized poloxamer of the method is FG-(poloxamer 188)-FG, FG-(poloxamer 237)-FG, FG-(poloxamer 338)-FG, FG-(poloxamer 407)-FG, or a combination of any of them.

In certain embodiments, independently for each occurrence the amine moiety of the linker-amine moiety of the method is 1,2-diaminoethane, tris (2-aminoethyl) amine, or a low molecular weight (e.g., less than about 2,000, 1,000, 500, or 250 Daltons) polyethyleneimine. For example, the amine moiety of the linker-amine moiety is a 1, 2-diaminoethane residue, a tris (2-aminoethyl) amine residue, or a combination thereof. In certain embodiments, the amine moiety of the linker-amine moiety is provided by a modification of a poloxamer with 1,2-diaminoethane, tris (2-aminoethyl) amine, or a combination thereof.

In certain embodiments, the oxidized polysaccharide of the method has a molecular weight of about 1,000 Da to about 100,000 Da. Unless specified otherwise, the “molecular weight” of the polysaccharide refers to the number average molecular weight.

In certain embodiments, the degree of functionalization of the oxidized polysaccharide of the method is about 10% to about 75%.

In certain embodiments, the oxidized polysaccharide of the method comprises 2 to 100 aldehyde moieties.

In certain embodiments, the oxidized polysaccharide comprises 40 to 80 aldehyde moieties.

In certain embodiments, the oxidized polysaccharide comprises 50 to 70 aldehyde moieties.

In certain embodiments, the oxidized polysaccharide of the method is a dextran.

In certain embodiments, the dextran has a molecular weight of about 10 kDa.

In certain embodiments, about 50% of the hydroxyl groups of the dextran are oxidized to aldehydes.

In certain embodiments, curing of the adhesive formulation in contact with the one or more biological tissues of the method comprises covalent bonding, ionic bonding, mechanical interlocking, or a combination thereof.

In certain embodiments, the adhesive formulation has a gelation time of about 1 second to about 100 seconds. Gelation time is the time that the adhesive formulation takes to become a gel. A gel is considered formed when the material does not flow.

In certain embodiments, the adhesive formulation has a gelation time of about 40 seconds to about 80 seconds.

In certain embodiments, the adhesive formulation has a swelling value of about 30% to about 90%. The swelling value is the material mass increase due to fluid penetration into the void spaces between the polymeric chains of the adhesive formulation.

In certain embodiments, the adhesive formulation has a swelling value of about 40% to about 75%.

In certain embodiments, the first component and the second component of the method are combined on the surface of the one or more biological tissues; and the adhesive formulation is formed on the surface of the one or more biological tissues; optionally wherein the adhesive formulation is formed on a suture line associated with a PTFE or polyester vascular graft.

In certain embodiments, the one or more biological tissues of the method comprise human tissue.

In certain embodiments, the first component, second component, or both further comprise an additive selected from the group consisting of foaming agents, pH modifiers, thickeners, antimicrobial agents, colorants, surfactants, and radio-opaque agents.

In certain embodiments, the first component is an aqueous solution.

In certain embodiments, the second component is an aqueous solution.

In certain embodiments, the first component, second component, or both components, comprise an effective amount of at least one drug.

In certain embodiments, the method further comprises the step of permitting the at least one drug to diffuse from the adhesive formulation into the biological tissue.

In certain embodiments, the adhesive formulation of the method reduces inflammation, enhances healing, or both in the biological tissue.

In certain embodiments, the biological tissue is a gastrointestinal (GI) tissue.

In certain embodiments, the first component and the second component are provided using an endoscopic technique.

In an aspect, disclosed is a kit, comprising:
a first component, comprising a block copolymer comprising one or more polyethylene oxide (PEO) blocks, one or more polypropylene oxide (PPO) blocks, and at least one primary amine group; and
a second component, comprising an oxidized polysaccharide comprising at least two aldehyde moieties;
a first syringe; and
instructions for use.

In an aspect, disclosed is a kit, comprising:
a first component, comprising a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine; and
a second component, comprising an oxidized polysaccharide comprising at least two aldehyde moieties;
a first syringe; and
instructions for use.

In certain embodiments, the first syringe of the kit comprises a first reservoir and a second reservoir; the first reservoir comprises the first component; and the second reservoir comprises the second component; optionally the first syringe comprises a mixing tip or a spray nozzle.

In certain embodiments, the kit further comprises a second syringe.

In certain embodiments, the first component of the kit is stored in the first syringe, and the second component is stored in the second syringe.

In certain embodiments, each of the first syringe and the second syringe of the kit comprise a mixing tip or a spray nozzle.

In certain embodiments, the instructions of the kit explain how to select an appropriate concentration of the first component or the second component or both to compensate for the characteristics of one or more biological tissues.

In certain embodiments, the first component or the second component or both comprise a drug.

In an aspect, disclosed is a drug delivery composition, comprising:
a biocompatible adhesive material disclosed herein; and
an effective amount of at least one drug;
wherein the at least one drug is admixed with the biocompatible adhesive material

In certain embodiments, the drug delivery composition is in contact with a biological tissue.

Generally, the adhesive material may be used on any internal or external biological tissues. The biological tissues may be human or other mammalian tissue. The biological tissues may be natural or artificially generated. The biological tissues may be skin, bone, ocular, muscular, vascular, dura or an internal organ, such as lung, intestine, heart, liver, etc. The adhesive formulation can be applied to a tissue site in a human or other animal patient, for example, during a surgical or other medical procedure. In one embodiment, the adhesive formulation is used to seal an anastomosis. In some embodiment, the adhesive formulation is used to adhere, seal, and/or treat a wound, lesion, or a combination thereof. For example, the adhesive formulation may be applied to slow-healing or troublesome wounds, such as those suffered by diabetics.

Generally, the adhesive materials and formulations disclosed herein may be used on any internal or external biological tissues. The biological tissues may be human or other mammalian tissue. The biological tissues may be natural or artificially generated. The biological tissues may be skin, bone, ocular, muscular, vascular, or an internal organ, such as lung, intestine, heart, liver, etc.

The adhesive material and/or formulation can be applied to a tissue site in a human or other animal patient, for example, during a surgical or other medical procedure. In one embodiment, the adhesive material is used to seal an anastomosis. In some embodiment, the adhesive formulation is used to adhere, seal, and/or treat a wound, lesion, or a combination thereof. For example, the adhesive formulation may be applied to slow-healing or troublesome wounds, such as those suffered by diabetics. In one embodiment, the adhesive formulation may be used to secure or help secure a medical implant, such as an orthopedic implant, within a human or other animal patient. In some embodiment, the adhesive formulation is used to seal and protect lesions in the gastrointestinal tract such as peptic ulcers or polyp resection lacerations.

### Poloxamer Component

Generally, the poloxamer may be made using any known methods. In one embodiment, the poloxamer is made by treating a starting poloxamer having end groups comprising at least one hydroxyl group (for example, Pluronic F-68) with a polyfunctional amine (for example, Polyethyleneimine) and a coupling agent (for example, 1,1′-Carbonyldiimidazole (CDI)) so that at least a portion of the surface groups comprise at least one amine.

In certain embodiments, the poloxamer component or first component is combined with a liquid to form a poloxamer component (a first component) solution. In one embodiment, the poloxamer component solution is an aqueous solution. In one embodiment, the solution comprises water, phosphate buffer saline (PBS), Dulbecco's Modified Eagle's Medium (DMEM), borate buffer, carbonate buffer, or any combination thereof. In one embodiment, the poloxamer component concentration in the poloxamer component solution is about 5% to about 25% by weight. In another embodiment, the poloxamer component concentration in the poloxamer component solution is about 10% to about 20% by weight. In a further embodiment, the poloxamer component concentration in the poloxamer component solution is about 11% to about 15% by weight.

In some instances, the poloxamer component or poloxamer component solution may further comprise an additive. Generally, the amount of additive may vary depending on the application, tissue type, concentration of the poloxamer component solution, the type of poloxamer component, concentration of the oxidized polysaccharide component solutions, and/or the type of oxidized polysaccharide component. Example of suitable additives, include but are not limited to, pH modifiers, thickeners, antimicrobial agents, colorants, surfactants, and radio-opaque compounds. Specific examples of these types of additives are described herein. In one embodiment, the poloxamer component solution comprises a foaming additive.

In particular embodiments, the poloxamer component or poloxamer component solution comprises an effective amount of at least one drug. In such embodiments, the adhesive formulation may serve as a matrix material for controlled release of drug. The drug may be essentially any drug suitable for local, regional, or systemic administration from a quantity of the adhesive formulation that has been applied to one or more tissue sites in a patient. In one embodiment, the drug comprises a thrombogenic agent. Non-limiting examples of thrombogenic agents include thrombin, fibrinogen, homocysteine, estramustine, and combinations thereof. In another embodiment, the drug comprises an anti-inflammatory agent. Non-limiting examples of anti-inflammatory agents include indomethacin, salicyclic acid acetate, ibuprophen, sulindac, piroxicam, naproxen, and combinations thereof. In still another embodiment, the drug comprises an anti-neoplastic agent. In still other embodiments, the drug is one for gene therapy. For example, the drug may comprise siRNA molecules to combat cancer. Other drugs are envisioned.

In other particular embodiments, the poloxamer component or poloxamer component solution comprises one or more cells. For example, the adhesive formulation may serve as a matrix material for delivering cells to a tissue site at which the adhesive formulation has been applied. In embodiments, the cells may comprise endothelial cells (EC), endothelial progenitor cells (EPC), hematopoietic stem cells, or other stem cells. In one embodiment, the cells are capable of releasing factors to treat cardiovascular disease and/or to reduce restenosis. Other types of cells are envisioned.

### Oxidized Polysaccharide Component

Generally, the oxidized polysaccharide component or second component comprises an oxidized polysaccharide with one or more functional groups capable of reacting with one or more functional groups on a biological tissue and/or one or more functional groups on the poloxamer component. In these embodiments, the at least one polysaccharide may be linear, branched, or have both linear and branched sections within its structure. Generally, the at least one polysaccharide may be natural, synthetic, or modified—for example, by cross-linking, altering the polysaccharide's substituents, or both. In one embodiment, the at least one polysaccharide is plant-based. In another embodiment, the at least one polysaccharide is animal-based. In another embodiment, the at least one polysaccharide is bacterial-derived. In yet another embodiment, the at least one polysaccharide is a combination of plant-based, animal-based, or bacterial-derived polysaccharides. Non-limiting examples of polysaccharides include, but are not limited to, dextran, chitin, starch, agar, cellulose, hyaluronic acid, or a combination thereof.

In some embodiments, the oxidized polysaccharide includes one or more functional groups that will react with one or more functional groups on a biological tissue and/or one or more functional groups on the poloxamer component. In one embodiment, at least two or more functional groups incorporated into the oxidized polysaccharide's structure is aldehyde.

In certain embodiments, the oxidized polysaccharide's degree of functionalization is adjustable. The “degree of functionalization” generally refers to the number or percentage of reactive groups on the oxidized polysaccharide that are replaced or converted to the desired one or more functional groups. In one embodiment, the degree of functionalization is adjusted based on the type of tissue to which the adhesive is applied, the concentration(s) of the components, and/or the type of oxidized polysaccharide or poloxamer used in the adhesive. In one embodiment, the degree of functionalization is from about 10% to about 75%. In another embodiment, the degree of functionalization is from about 15% to about 50%. In yet another embodiment, the degree of functionalization is from about 20% to about 30%.

In some embodiments, an oxidized polysaccharide includes a desired percentage of two or more aldehyde functional groups. Generally, this oxidation may be conducted using any known means. For example, suitable oxidizing agents include, but are not limited to, periodates, hypochlorites, ozone, peroxides, hydroperoxides, persulfates, and percarbonates. In one embodiment, the oxidation is performed using sodium periodate. Typically, different amounts of oxidizing agents may be used to alter the degree of functionalization.

In certain embodiments, the oxidized polysaccharide component or second component is combined with a liquid to form an oxidized polysaccharide component solution or a second component solution. In one embodiment, the oxidized polysaccharide component solution is an aqueous solution. In one embodiment, the solution comprises water, PBS, DMEM, or any combination thereof.

Generally, the oxidized polysaccharide component solution may have any suitable concentration of oxidized polysaccharide component. In one embodiment, the oxidized polysaccharide component concentration in the oxidized polysaccharide component solution is about 5% to about 40% by weight. In another embodiment, the oxidized polysaccharide component concentration in the oxidized polysaccharide component solution is about 5% to about 30% by weight. In yet another embodiment, the oxidized polysaccharide component concentration in the oxidized polysaccharide component solution is about 5% to about 25% by weight. Typically, the concentration may be tailored and/or adjusted based on the particular application, tissue type, and/or the type and concentration of poloxamer component used.

The oxidized polysaccharide component or oxidized polysaccharide component solution may also comprise one or more additives. In one embodiment, the additive is compatible with the oxidized polysaccharide component. In another embodiment, the additive does not contain primary or secondary amines. Generally, the amount of additive varies depending on the application, tissue type, concentration of the oxidized polysaccharide component solution, the type of oxidized polysaccharide component and/or poloxamer component. Examples of suitable additives, include, but are not limited to, pH modifiers, thickeners, antimicrobial agents, colorants, surfactants, and radio-opaque compounds. In other embodiments, the oxidized polysaccharide component solution comprises a foaming agent.

In certain embodiments, the pH modifier is an acidic compound. Examples of acidic pH modifiers include, but are not limited to, carboxylic acids, inorganic acids, and sulfonic acids. In other embodiments, the pH modifier is a basic compound. Examples of basic pH modifiers include, but are not limited to, hydroxides, alkoxides, nitrogen-containing compounds other than primary and secondary amines, basic carbonates, and basic phosphates.

Generally, the thickener may be selected from any known viscosity-modifying compounds, including, but not limited to, polysaccharides and derivatives thereof, such as starch or hydroxyethyl cellulose.

Generally, the surfactant may be any compound that lowers the surface tension of water. In one embodiment, the surfactant is an ionic surfactant—for example, sodium lauryl sulfate. In another embodiment, the surfactant is a neutral surfactant. Examples of neutral surfactants include, but are not limited to, polyoxyethylene ethers, polyoxyethylene esters, and polyoxyethylene sorbitan.

In one embodiment, the radio-opaque compound is barium sulfate, gold particles, or a combination thereof.

In particular embodiments, the oxidized polysaccharide component or oxidized polysaccharide component solution comprises an effective amount of at least one drug. In such embodiments, the adhesive formulation may serve as a matrix material for controlled release of drug. The drug may be essentially any drug suitable for local, regional, or systemic administration from a quantity of the adhesive formulation that has been applied to one or more tissue sites in a patient. In one embodiment, the drug comprises a thrombogenic agent. Non-limiting examples of thrombogenic agents include thrombin, fibrinogen, homocysteine, estramustine, and combinations thereof. In another embodiment, the drug comprises an anti-inflammatory agent. Non-limiting examples of anti-inflammatory agents include indomethacin, salicyclic acid acetate, ibuprophen, sulindac, piroxicam, naproxen, and combinations thereof. In still another embodiment, the drug comprises an anti-neoplastic agent. In still other embodiments, the drug is one for gene or cell therapy. For example, the drug may comprise siRNA molecules to combat cancer. Other drugs are envisioned.

In other particular embodiments, the oxidized polysaccharide component or oxidized polysaccharide component solution comprises one or more cells. For example, the adhesive formulation may serve as a matrix material for delivering cells to a tissue site at which the adhesive formulation has been applied. In embodiments, the cells may comprise endothelial cells (EC), endothelial progenitor cells (EPC), hematopoietic stem cells, or other stem cells. In one embodiment, the cells are capable of releasing factors to treat cardiovascular disease and/or to reduce restenosis. Other types of cells are envisioned.

### Biologically Active Agents and Pharmaceutically Active Agents

In certain embodiments, one or more biologically active agents may be incorporated in the biocompatible adhesive materials of the invention. Active agents amenable for use in the compositions of the present invention include growth factors, such as transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the transforming growth factor (TGF) supergene family, which are multifunctional regulatory proteins, are particularly preferred. Members of the TGF supergene family include the beta transforming growth factors (for example, TGF-β1, TGF-B2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

In addition to the biological active agents discussed above, a large number of pharmaceutical agents are known in the art and are amenable for use in the biocompatible adhesive materials of the invention. The term “pharmaceutical agent” includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Non-limiting examples of broad categories of useful pharmaceutical agents include the following therapeutic categories: anabolic agents, antacids, anti-asthmatic agents, anti-cholesterolemic and anti-lipid agents, anti-coagulants, anti-convulsants, anti-diarrheals, anti-emetics, anti-infective agents, anti-inflammatory agents, anti-manic agents, anti-nauseants, anti-neoplastic agents, anti-obesity agents, anti-pyretic and analgesic agents, anti-spasmodic agents, anti-thrombotic agents, anti-uricemic agents, anti-anginal agents, antihistamines, anti-tussives, appetite suppressants, biologicals, cerebral dilators, coronary dilators, decongestants, diuretics, diagnostic agents, erythropoietic agents, expectorants, gastrointestinal sedatives, hyperglycemic agents, hypnotics, hypoglycemic agents, ion exchange resins, laxatives, mineral supplements, mucolytic agents, neuromuscular drugs, peripheral vasodilators, psychotropics, sedatives, stimulants, thyroid and anti-thyroid agents, uterine relaxants, vitamins, and prodrugs.

More specifically, non-limiting examples of useful pharmaceutical agents include the following therapeutic categories: analgesics, such as nonsteroidal anti-inflammatory drugs, opiate agonists and salicylates; antihistamines, such as H1-blockers and H2-blockers; anti-infective agents, such as anthelmintics, antianaerobics, antibiotics, aminoglycoside antibiotics, antifungal antibiotics, cephalosporin antibiotics, macrolide antibiotics, miscellaneous beta-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, and urinary anti-infectives; antineoplastic agents, such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics; autonomic agents, such as anticholinergics, antimuscarinic anticholinergics, ergot alkaloids, parasympathomimetics, cholinergic agonist parasympathomimetics, cholinesterase inhibitor para-sympathomimetics, sympatholytics, alpha-blocker sympatholytics, beta-blocker sympatholytics, sympathomimetics, and adrenergic agonist sympathomimetics; cardiovascular agents, such as antianginals, beta-blocker antianginals, calcium-channel blocker antianginals, nitrate antianginals, antiarrhythmics, cardiac glycoside antiarrhythmics, class I antiarrhythmics, class II antiarrhythmics, class III antiarrhythmics, class IV antiarrhythmics, antihypertensive agents, alpha-blocker antihypertensives, angiotensin-converting enzyme inhibitor (ACE inhibitor) antihypertensives, beta-blocker antihypertensives, calcium-channel blocker antihypertensives, central-acting adrenergic antihypertensives, diuretic antihypertensive agents, peripheral vasodilator antihypertensives, antilipemics, bile acid sequestrant antilipemics, HMG-COA reductase inhibitor antilipemics, inotropes, cardiac glycoside inotropes, and thrombolytic agents; dermatological agents, such as antihistamines, anti-inflammatory agents, corticosteroid anti-inflammatory agents, antipruritics/local anesthetics, topical anti-infectives, antifungal topical anti-infectives, antiviral topical anti-infectives, and topical antineoplastics; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hematological agents, such as antianemia agents, hematopoietic antianemia agents, coagulation agents, anticoagulants, hemostatic coagulation agents, platelet inhibitor coagulation agents, thrombolytic enzyme coagulation agents, and plasma volume expanders; hormones and hormone modifiers, such as abortifacients, adrenal agents, corticosteroid adrenal agents, androgens, anti-androgens, antidiabetic agents, sulfonylurea antidiabetic agents, antihypoglycemic agents, oral contraceptives, progestin contraceptives, estrogens, fertility agents, oxytocics, parathyroid agents, pituitary hormones, progestins, antithyroid agents, thyroid hormones, and tocolytics; immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immuno-suppressive anti-inflammatory agents, nonsteroidal anti-inflammatory drugs (NSAIDs), salicylate anti-inflammatory agents, skeletal muscle relaxants, neuromuscular blocker skeletal muscle relaxants, and reverse neuromuscular blocker skeletal muscle relaxants; neurological agents, such as anticonvulsants, barbiturate anticonvulsants, benzodiazepine anticonvulsants, anti-migraine agents, anti-parkinsonian agents, anti-vertigo agents, opiate agonists, and opiate antagonists; ophthalmic agents, such as anti-glaucoma agents, beta-blocker anti-gluacoma agents, miotic anti-glaucoma agents, mydriatics, adrenergic agonist mydriatics, antimuscarinic mydriatics, ophthalmic anesthetics, ophthalmic anti-infectives, ophthalmic aminoglycoside anti-infectives, ophthalmic macrolide anti-infectives, ophthalmic quinolone anti-infectives, ophthalmic sulfonamide anti-infectives, ophthalmic tetracycline anti-infectives, ophthalmic anti-inflammatory agents, ophthalmic corticosteroid anti-inflammatory agents, and ophthalmic nonsteroidal anti-inflammatory drugs (NSAIDs); psychotropic agents, such as antidepressants, heterocyclic antidepressants, monoamine oxidase inhibitors (MAOIs), selective serotonin re-uptake inhibitors (SSRIs), tricyclic antidepressants, antimanics, antipsychotics, phenothiazine antipsychotics, anxiolytics, sedatives, and hypnotics, barbiturate sedatives and hypnotics, benzodiazepine anxiolytics, sedatives, and hypnotics, and psychostimulants; respiratory agents, such as antitussives, bronchodilators, adrenergic agonist bronchodilators, antimuscarinic bronchodilators, expectorants, mucolytic agents, respiratory anti-inflammatory agents, and respiratory corticosteroid anti-inflammatory agents; toxicology agents, such as antidotes, heavy metal antagonists/chelating agents, substance abuse agents, deterrent substance abuse agents, and withdrawal substance abuse agents; minerals; and vitamins, such as vitamin A, vitamin B, vitamin C, vitamin D, vitamin E, and vitamin K.

Preferred classes of useful pharmaceutical agents from the above categories include: (1) nonsteroidal anti-inflammatory drugs (NSAIDs) analgesics, such as diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) H1-blocker antihistamines, such as clemastine and terfenadine; (5) H2-blocker antihistamines, such as cimetidine, famotidine, nizadine, and ranitidine; (6) anti-infective agents, such as mupirocin; (7) antianaerobic anti-infectives, such as chloramphenicol and clindamycin; (8) antifungal antibiotic anti-infectives, such as amphotericin b, clotrimazole, fluconazole, and ketoconazole; (9) macrolide antibiotic anti-infectives, such as azithromycin and erythromycin; (10) miscellaneous beta-lactam antibiotic anti-infectives, such as aztreonam and imipenem; (11) penicillin antibiotic anti-infectives, such as nafcillin, oxacillin, penicillin G, and penicillin V; (12) quinolone antibiotic anti-infectives, such as ciprofloxacin and norfloxacin; (13) tetracycline antibiotic anti-infectives, such as doxycycline, minocycline, and tetracycline; (14) antituberculosis antimycobacterial anti-infectives such as isoniazid (INH), and rifampin; (15) antiprotozoal anti-infectives, such as atovaquone and dapsone; (16) antimalarial antiprotozoal anti-infectives, such as chloroquine and pyrimethamine; (17) anti-retroviral anti-infectives, such as ritonavir and zidovudine; (18) antiviral anti-infective agents, such as acyclovir, ganciclovir, interferon alfa, and rimantadine; (19) alkylating antineoplastic agents, such as carboplatin and cisplatin; (20) nitrosourea alkylating antineoplastic agents, such as carmustine (BCNU); (21) antimetabolite antineoplastic agents, such as methotrexate; (22) pyrimidine analog antimetabolite antineoplastic agents, such as fluorouracil (5-FU) and gemcitabine; (23) hormonal antineoplastics, such as goserelin, leuprolide, and tamoxifen; (24) natural antineoplastics, such as aldesleukin, interleukin-2, docetaxel, etoposide (VP-16), interferon alfa, paclitaxel, and tretinoin (ATRA); (25) antibiotic natural antineoplastics, such as bleomycin, dactinomycin, daunorubicin, doxorubicin, and mitomycin; (26) vinca alkaloid natural antineoplastics, such as vinblastine and vincristine; (27) autonomic agents, such as nicotine; (28) anticholinergic autonomic agents, such as benztropine and trihexyphenidyl; (29) antimuscarinic anticholinergic autonomic agents, such as atropine and oxybutynin; (30) ergot alkaloid autonomic agents, such as bromocriptine; (31) cholinergic agonist parasympathomimetics, such as pilocarpine; (32) cholinesterase inhibitor parasympathomimetics, such as pyridostigmine; (33) alpha-blocker sympatholytics, such as prazosin; (34) beta-blocker sympatholytics, such as atenolol; (35) adrenergic agonist sympathomimetics, such as albuterol and dobutamine; (36) cardiovascular agents, such as aspirin (ASA) (enteric coated ASA); (37) beta-blocker antianginals, such as atenolol and propranolol; (38) calcium-channel blocker antianginals, such as nifedipine and verapamil; (39) nitrate antianginals, such as isosorbide dinitrate (ISDN); (40) cardiac glycoside antiarrhythmics, such as digoxin; (41) class I anti-arrhythmics, such as lidocaine, mexiletine, phenytoin, procainamide, and quinidine; (42) class II antiarrhythmics, such as atenolol, metoprolol, propranolol, and timolol; (43) class III antiarrhythmics, such as amiodarone; (44) class IV antiarrhythmics, such as diltiazem and verapamil; (45) a-blocker antihypertensives, such as prazosin; (46) angiotensin-converting enzyme inhibitor (ACE inhibitor) antihypertensives, such as captopril and enalapril; (47) β-blocker antihypertensives, such as atenolol, metoprolol, nadolol, and propanolol; (48) calcium-channel blocker antihypertensive agents, such as diltiazem and nifedipine; (49) central-acting adrenergic antihypertensives, such as clonidine and methyldopa; (50) diurectic antihypertensive agents, such as amiloride, furosemide, hydrochlorothiazide (HCTZ), and spironolactone; (51) peripheral vasodilator antihypertensives, such as hydralazine and minoxidil; (52) antilipemics, such as gemfibrozil and probucol; (53) bile acid sequestrant antilipemics, such as cholestyramine; (54) HMG-CoA reductase inhibitor antilipemics, such as lovastatin and pravastatin; (55) inotropes, such as amrinone, dobutamine, and dopamine; (56) cardiac glycoside inotropes, such as digoxin; (57) thrombolytic agents, such as alteplase (TPA), anistreplase, streptokinase, and urokinase; (58) dermatological agents, such as colchicine, isotretinoin, methotrexate, minoxidil, tretinoin (ATRA); (59) dermatological corticosteroid anti-inflammatory agents, such as betamethasone and dexamethasone; (60) antifungal topical anti-infectives, such as amphotericin B, clotrimazole, miconazole, and nystatin; (61) antiviral topical anti-infectives, such as acyclovir; (62) topical antineoplastics, such as fluorouracil (5-FU); (63) electrolytic and renal agents, such as lactulose; (64) loop diuretics, such as furosemide; (65) potassium-sparing diuretics, such as triamterene; (66) thiazide diuretics, such as hydro-chlorothiazide (HCTZ); (67) uricosuric agents, such as probenecid; (68) enzymes such as RNase and DNase; (69) thrombolytic enzymes, such as alteplase, anistreplase, streptokinase and urokinase; (70) antiemetics, such as prochlorperazine; (71) salicylate gastrointestinal anti-inflammatory agents, such as sulfasalazine; (72) gastric acid-pump inhibitor anti-ulcer agents, such as omeprazole; (73) H2-blocker anti-ulcer agents, such as cimetidine, famotidine, nizatidine, and ranitidine; (74) digestants, such as pancrelipase; (75) prokinetic agents, such as erythromycin; (76) opiate agonist intravenous anesthetics such as fentanyl; (77) hematopoietic antianemia agents, such as erythropoietin, filgrastim (G-CSF), and sargramostim (GM-CSF); (78) coagulation agents, such as antihemophilic factors 1-10 (AHF 1-10); (79) anticoagulants, such as warfarin; (80) thrombolytic enzyme coagulation agents, such as alteplase, anistreplase, streptokinase and urokinase; (81) hormones and hormone modifiers, such as bromocriptine; (82) abortifacients, such as methotrexate; (83) antidiabetic agents, such as insulin; (84) oral contraceptives, such as estrogen and progestin; (85) progestin contraceptives, such as levonorgestrel and norgestrel; (86) estrogens such as conjugated estrogens, diethylstilbestrol (DES), estrogen (estradiol, estrone, and estropipate); (87) fertility agents, such as clomiphene, human chorionic gonadatropin (HCG), and menotropins; (88) parathyroid agents such as calcitonin; (89) pituitary hormones, such as desmopressin, goserelin, oxytocin, and vasopressin (ADH); (90) progestins, such as medroxyprogesterone, norethindrone, and progesterone; (91) thyroid hormones, such as levothyroxine; (92) immunobiologic agents, such as interferon beta-1b and interferon gamma-1b; (93) immunoglobulins, such as immune globulin IM, IMIG, IGIM and immune globulin IV, IVIG, IGIV; (94) amide local anesthetics, such as lidocaine; (95) ester local anesthetics, such as benzocaine and procaine; (96) musculoskeletal corticosteroid anti-inflammatory agents, such as beclomethasone, betamethasone, cortisone, dexamethasone, hydrocortisone, and prednisone; (97) musculoskeletal anti-inflammatory immunosuppressives, such as azathioprine, cyclophosphamide, and methotrexate; (98) musculoskeletal nonsteroidal anti-inflammatory drugs (NSAIDs), such as diclofenac, ibuprofen, ketoprofen, ketorlac, and naproxen; (99) skeletal muscle relaxants, such as baclofen, cyclobenzaprine, and diazepam; (100) reverse neuromuscular blocker skeletal muscle relaxants, such as pyridostigmine; (101) neurological agents, such as nimodipine, riluzole, tacrine and ticlopidine; (102) anticonvulsants, such as carbamazepine, gabapentin, lamotrigine, phenytoin, and valproic acid; (103) barbiturate anticonvulsants, such as phenobarbital and primidone; (104) benzodiazepine anticonvulsants, such as clonazepam, diazepam, and lorazepam; (105) anti-parkisonian agents, such as bromocriptine, levodopa, carbidopa, and pergolide; (106) anti-vertigo agents, such as meclizine; (107) opiate agonists, such as codeine, fentanyl, hydromorphone, methadone, and morphine; (108) opiate antagonists, such as naloxone; (109) β-blocker anti-glaucoma agents, such as timolol; (110) miotic anti-glaucoma agents, such as pilocarpine; (111) ophthalmic aminoglycoside antiinfectives, such as gentamicin, neomycin, and tobramycin; (112) ophthalmic quinolone anti-infectives, such as ciprofloxacin, norfloxacin, and ofloxacin; (113) ophthalmic corticosteroid anti-inflammatory agents, such as dexamethasone and prednisolone; (114) ophthalmic nonsteroidal anti-inflammatory drugs (NSAIDs), such as diclofenac; (115) antipsychotics, such as clozapine, haloperidol, and risperidone; (116) benzodiazepine anxiolytics, sedatives and hypnotics, such as clonazepam, diazepam, lorazepam, oxazepam, and prazepam; (117) psychostimulants, such as methylphenidate and pemoline; (118) antitussives, such as codeine; (119) bronchodilators, such as theophylline; (120) adrenergic agonist bronchodilators, such as albuterol; (121) respiratory corticosteroid anti-inflammatory agents, such as dexamethasone; (122) antidotes, such as flumazenil and naloxone; (123) heavy metal antagonists/chelating agents, such as penicillamine; (124) deterrent substance abuse agents, such as disulfiram, naltrexone, and nicotine; (125) withdrawal substance abuse agents, such as bromocriptine; (126) minerals, such as iron, calcium, and magnesium; (127) vitamin B compounds, such as cyanocobalamin (vitamin B12) and niacin (vitamin B3); (128) vitamin C compounds, such as ascorbic acid; and (129) vitamin D compounds, such as calcitriol.

In addition to the foregoing, the following less common drugs may also be used: chlorhexidine; estradiol cypionate in oil; estradiol valerate in oil; flurbiprofen; flurbiprofen sodium; ivermectin; levodopa; nafarelin; and somatropin. Further, the following drugs may also be used: recombinant beta-glucan; bovine immunoglobulin concentrate; bovine superoxide dismutase; the formulation comprising fluorouracil, epinephrine, and bovine collagen; recombinant hirudin (r-Hir), HIV-1 immunogen; human anti-TAC antibody; recombinant human growth hormone (r-hGH); recombinant human hemoglobin (r-Hb); recombinant human mecasermin (r-IGF-1); recombinant interferon β-la; lenograstim (G-CSF); olanzapine; recombinant thyroid stimulating hormone (r-TSH); and topotecan.

Further still, the following intravenous products may be used: acyclovir sodium; aldesleukin; atenolol; bleomycin sulfate, human calcitonin; salmon calcitonin; carboplatin; carmustine; dactinomycin, daunorubicin HCl; docetaxel; doxorubicin HCl; epoetin alfa; etoposide (VP-16); fluorouracil (5-FU); ganciclovir sodium; gentamicin sulfate; interferon alfa; leuprolide acetate; meperidine HCl; methadone HCl; methotrexate sodium; paclitaxel; ranitidine HCl; vinblastin sulfate; and zidovudine (AZT).

Further specific examples of useful pharmaceutical agents from the above categories include: (a) anti-neoplastics such as androgen inhibitors, antimetabolites, cytotoxic agents, and immunomodulators; (b) anti-tussives such as dextromethorphan, dextromethorphan hydrobromide, noscapine, carbetapentane citrate, and chlorphedianol hydrochloride; (c) antihistamines such as chlorpheniramine maleate, phenindamine tartrate, pyrilamine maleate, doxylamine succinate, and phenyltoloxamine citrate; (d) decongestants such as phenylephrine hydrochloride, phenylpropanolamine hydrochloride, pseudoephedrine hydrochloride, and ephedrine; (e) various alkaloids such as codeine phosphate, codeine sulfate and morphine; (f) mineral supplements such as potassium chloride, zinc chloride, calcium carbonates, magnesium oxide, and other alkali metal and alkaline earth metal salts; (g) ion exchange resins such as cholestryramine; (h) anti-arrhythmics such as N-acetylprocainamide; (i) antipyretics and analgesics such as acetaminophen, aspirin and ibuprofen; (j) appetite suppressants such as phenyl-propanolamine hydrochloride or caffeine; (k) expectorants such as guaifenesin; (1) antacids such as aluminum hydroxide and magnesium hydroxide; (m) biologicals such as peptides, polypeptides, proteins and amino acids, hormones, interferons or cytokines, and other bioactive peptidic compounds, such as interleukins 1-18 including mutants and analogues, RNase, DNase, luteinizing hormone releasing hormone (LHRH) and analogues, gonadotropin releasing hormone (GnRH), transforming growth factor-. beta. (TGF-beta), fibroblast growth factor (FGF), tumor necrosis factor-alpha & beta (TNF-alpha & beta), nerve growth factor (NGF), growth hormone releasing factor (GHRF), epidermal growth factor (EGF), fibroblast growth factor homologous factor (FGFHF), hepatocyte growth factor (HGF), insulin growth factor (IGF), invasion inhibiting factor-2 (IIF-2), bone morphogenetic proteins 1-7 (BMP 1-7), somatostatin, thymosin-alpha-1, gamma-globulin, superoxide dismutase (SOD), complement factors, hGH, tPA, calcitonin, ANF, EPO and insulin; and (n) anti-infective agents such as antifungals, anti-virals, antiseptics and antibiotics.

Alternatively, the pharmaceutical agent may be a radiosensitizer, such as metoclopramide, sensamide or neusensamide (manufactured by Oxigene); profiromycin (made by Vion); RSR13 (made by Allos); Thymitaq (made by Agouron), etanidazole or lobenguane (manufactured by Nycomed); gadolinium texaphrin (made by Pharmacyclics); BuDR/Broxine (made by NeoPharm); IPdR (made by Sparta); CR2412 (made by Cell Therapeutic); LIX (made by Terrapin); or the like. Preferably, the biologically active substance is selected from the group consisting of peptides, poly-peptides, proteins, amino acids, polysaccharides, growth factors, hormones, anti-angiogenesis factors, interferons or cytokines, and pro-drugs. In a particularly preferred embodiment, the biologically active substance is a therapeutic drug or pro-drug, most preferably a drug selected from the group consisting of chemotherapeutic agents and other anti-neoplastics such as paclitaxel, antibiotics, anti-virals, antifungals, anti-inflammatoires, and anticoagulants.

The biologically active substances are used in amounts that are therapeutically effective. While the effective amount of a biologically active substance will depend on the particular material being used, amounts of the biologically active substance from about 1% to about 65% may be desirable. Lesser amounts may be used to achieve efficacious levels of treatment for certain biologically active substances.

### Adhesive Material or Adhesive Formulation

Generally, the adhesive material or adhesive formulations described herein may be formed by combining the oxidized polysaccharide component or oxidized polysaccharide component solution, and the poloxamer component or poloxamer component solution in any manner. In some embodiments, the oxidized polysaccharide component or oxidized polysaccharide component solution, and the poloxamer component or poloxamer component solution are combined before contacting a biological tissue with the adhesive formulation. In other embodiments, the oxidized polysaccharide component or oxidized polysaccharide component solution, and the poloxamer component or poloxamer component solution are combined, in any order, on a biological tissue. In further embodiments, the oxidized polysaccharide component or oxidized polysaccharide component solution is applied to a first biological tissue, the poloxamer component or poloxamer component solution is applied to a second biological tissue, and the first and second biological tissues are contacted. In still a further embodiment, the oxidized polysaccharide component or oxidized polysaccharide component solution is applied to a first region a biological tissue, the poloxamer component or poloxamer component solution is applied to a second region of a biological tissue, and the first and second regions are contacted.

Generally, the adhesive formulation may be applied to one or more biological tissues as an adhesive, sealant, and/or treatment. The one or more biological tissues may be diseased or healthy. In one embodiment, the adhesive formulation is applied to one or more biological tissues as an adhesive. In another embodiment, the adhesive formulation is applied to one or more biological tissues as a sealant. In a further embodiment, the adhesive formulation is applied to one or more biological tissues as a treatment. In an additional embodiment, the adhesive formulation is applied to one or more biological tissues as an adhesive and sealant. In still another embodiment, the adhesive formulation is applied to one or more biological tissues as an adhesive and treatment. In yet another embodiment, the adhesive formulation is applied to one or more biological tissues as a sealant and treatment. In a still further embodiment, the adhesive formulation is applied to one or more biological tissues as an adhesive, sealant, and treatment.

As used herein, the adhesive material or adhesive formulation is a “treatment” when it improves the response of at least one biological tissue to which it is applied. In some embodiments, the improved response is lessening overall inflammation, improving the specific response at the wound site/interface of the tissue and adhesive formulation, enhancing healing, or a combination thereof. As used herein, the phrase “lessening overall inflammation” refers to an improvement of histology scores that reflect the severity of inflammation. As used herein, the phrase “improving the specific response at the wound site/interface of the tissue and adhesive formulation” refers to an improvement of histology scores that reflect the severity of serosal neutrophils. As used herein, the phrase “enhancing healing” refers to an improvement of histology scores that reflect the severity of serosal fibrosis.

After contacting one or more biological tissues, the adhesive formulations may be allowed adequate time to cure or gel. When the adhesive formulation “cures” or “gels,” as those terms are used herein, it means that the reactive groups on the oxidized polysaccharide component, poloxamer component, and one or more biological tissues have undergone one or more reactions. Not wishing to be bound by any particular theory, it is believed that the adhesive formulations described herein are effective because the oxidized polysaccharide component reacts with both the poloxamer component and the surface of the biological tissues. In certain embodiments, the oxidized polysaccharide component's aldehyde functional groups react with the amines on the poloxamer component and the biological tissues to form imine bonds. In these embodiments, it is believed that the amines on the poloxamer component react with a high percentage of the aldehydes on the oxidized polysaccharide component, thereby reducing toxicity and increasing biocompatibility of the adhesive formulations. Typically, the time needed to cure or gel the adhesive formulations will vary based on a number of factors, including, but not limited to, the characteristics of the oxidized polysaccharide component and/or poloxamer component, the concentrations of the oxidized polysaccharide component solution and/or the poloxamer component solution, and the characteristics of the one or more biological tissues. In embodiments, the adhesive formulation will cure sufficiently to provide desired bonding or sealing shortly after the components are combined. The gelation or cure time should provide that a mixture of the components can be delivered in fluid form to a target area before becoming too viscous or solidified and then once applied to the target area sets up rapidly thereafter. In one embodiment, the gelation or cure time is less than 120 seconds. In another embodiment, the gelation or cure time is between 1 and 100 seconds. In a particular embodiment, the gelation or cure time is between 3 and 60 seconds.

### Adhesive Formulation Kits

In another aspect, a kit is provided that comprises a first part that includes a oxidized polysaccharide component or oxidized polysaccharide component solution, and a second part that includes a poloxamer component or poloxamer component solution. The kit may further include an applicator or other device means, such as a multi-compartment syringe, for storing, combining, and delivering the two parts and/or the resulting adhesive formulation to a tissue site.

In certain embodiments, the kit comprises at least one syringe. In one embodiment, the syringe comprises separate reservoirs for the oxidized polysaccharide component solution and the poloxamer component solution. The syringe may also comprise a mixing tip or spray nozzle or delivery catheter that combines the two solutions as the plunger is depressed. The mixing tip may be releasably securable to the syringe (to enable exchange of mixing tips), and the mixing tip may comprise a static mixer. In some embodiments, the reservoirs in the syringe may have different sizes or accommodate different volumes of solution. In other embodiments, the reservoirs in the syringe may be the same size or accommodate the same volumes of the solution. In a further embodiment, one reservoir may comprise Part 1 of the foaming composition described hereinabove, and a second reservoir may comprise Part 2 of the foaming composition.

In a further embodiment, one or more of the reservoirs of the syringe may be removable. In this embodiment, the removable reservoir may be replaced with a reservoir containing an oxidized polysaccharide component solution or a poloxamer component solution of a desired concentration.

In a preferred embodiment, the kit is sterile. For example, the components of the kit may be packaged together, for example in a tray, pouch, and/or box. The packaged kit may be sterilized using known techniques such as electron beam irradiation, gamma irradiation, ethylene oxide sterilization, or other suitable techniques.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

A “subject” to which administration is contemplated includes, but is not limited to, humans (i.e., a male or female of any age group, e.g., a pediatric subject (e.g., infant, child, adolescent) or adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment.

“Preventing” or “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “adhering” generally refers to affixing, permanently or temporarily, two or more biological tissues, or two or more regions of a biological tissue. As used herein, the term “sealing” generally refers to covering, at least partially, or filling, at least partially, one or more sites on one or more biological tissues, such as a wound. As used herein, the term “treating” generally refers to improving the response of at least one biological tissue to which one or more adhesive formulations is applied. In some embodiments, the “response” that is improved or enhanced includes inflammation, healing, or both.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

### Examples

In order that the invention described herein may be more fully understood, the following examples are set forth. The examples described in this application are offered to illustrate the compounds, compositions, materials, device, and methods provided herein and are not to be construed in any way as limiting their scope.

### Example 1: Amine-terminated poloxamer

The first component of the present disclosure consists of an aqueous solution of a homodifunctionalized amine terminated poloxamer. Poloxamers are lineal polymers with an A-B-A (FG-PEO—PPO-PEO-FG) structure, wherein FG independently for each occurrence represents H or a linker-amine moiety, at least one occurrence of FG is not H. The tri-block structure of the poloxamers provides this family of polymers with temperature sensitive properties.

Poloxamers are functionalized with different amine containing molecules providing a family of polymers with diverse number of amine terminal groups. Table 1 shows an example of some of the amines used to modify the poloxamers.

TABLE 1
Example of amine containing molecules
used to modify the poloxamer polymers.

| Amine containing molecule | Number of amines per molecule |
|---|---|
| 1,2-diaminoethane | 2 |
| Tris(2-aminoethyl)amine | 3 |
| Polyethyleneimine (LMW) | ~17 |

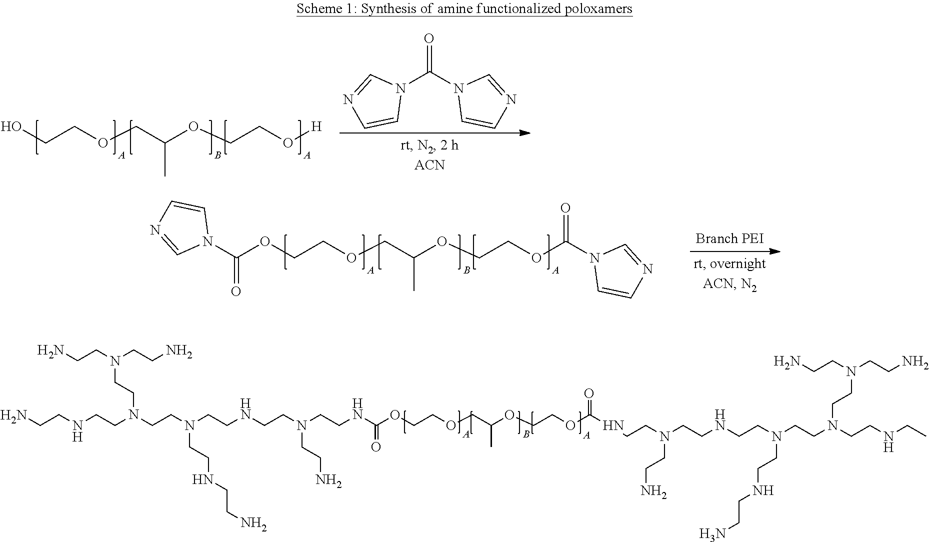

Poloxamers are functionalized with different amine containing molecules were synthesized as shown in Scheme 1 following the following synthetic procedure:
1. Dissolve 16.8g of Pluronic F-68 (2 mmol) in 50 mL of Acetonitrile (ACN)
2. Dissolve 3.25g (20 mmol) of 1,1′-Carbonyldiimidazole (CDI) in 40 mL of ACN and add it dropwise to the PluronicF-68 solution while stirring, maintaining an inert atmosphere. Allow the CDI to activate the Pluronic hydroxyl groups for 2 hours at room temperature.
3. In a round bottom flask dissolve 40g of Polyethyleneimine (PEI) in 40 mL of ACN. Add slowly (1 mL/min) dropwise the solution of the activated Pluronic over the stirring PEI. Allow it to react overnight at room temperature.
4. Evaporate the ACN (40° C./90mBar) from the reaction mixture and redissolve the obtained solid in 100 mL of H₂O.
5. Perform extensive dialysis against water to remove the unreacted PEI and CDI salts using spectrum membranes MWCO 3KDa. After 7 days of dialysis, freeze dry the purified solution to obtain the modified homobifunctional Plu-68-PEI.

To create a family of polymers with a broad range of biological and mechanical properties different types of poloxamer were used. In Table 2 some examples of poloxamers are displayed providing different molecular weight and hydrophilic/hydrophobic ratios.

TABLE 2
Example of nomenclature and properties of some of the
poloxamers used to create the adhesive hydrogel.

| | | Average | Average | Average | |
|---|---|---|---|---|---|
| | | number of | number of | molecular | |
| Pluronic | Poloxamer | EO units A | PO units B | weight | HBL |
| F87 | 237 | 122.5 | 39.83 | 7700 | >24 |
| F127 | 407 | 200.45 | 65.17 | 12600 | 18-23 |
| F68 | 188 | 152.73 | 28.97 | 8400 | >24 |
| F108 | 338 | 265.5 | 50.3 | 14600 | >24 |

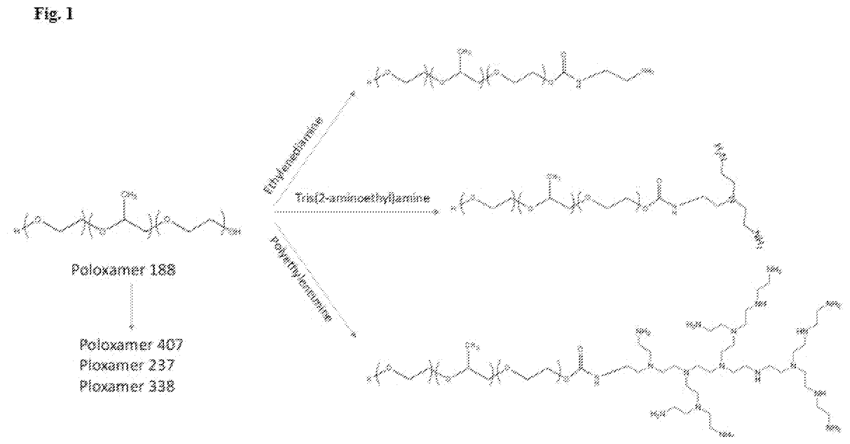

Different linear poloxamers (polyethylene oxide-polypropylene oxide-polyethylene oxide, PEO—PPO-PEO) polymers of multiple molecular weights and PEO: PPO ratios, have their terminal groups functionalized with polyamine molecules (FIG. 1). The different PEO: PPO ratios and total molecular weights, provide each of these polymers with a specific transition temperature between their different molecular clustering states.

### Example 2: Crosslinking agents

Polysaccharides such as dextran are oxidized to generate aldehyde groups along their backbone. If mixed at the appropriate pH, aldehydes present in the oxidized dextran can readily react with the terminal amines of the modified poloxamer. The reaction of the amine modified pluronic and oxidized dextran creates an adhesive, biodegradable, and biocompatible hydrogel. Aldehydes and amines form an imine bond which is stable but reversible. The reversibility of this chemical bond makes the material biodegradable and therefore internally implantable.

The aldehyde dextran is obtained by oxidation of 10 kDa dextran using sodium periodate. The amount of sodium periodate is adjusted to achieve an oxidation degree of ˜40-50%

### Example 3: Hydrogel synthesis with poloxamer 188

45% oxidized dextran is reacted with poloxamer 188 modified with low molecular weight polyethyleneimine. Oxidized dextran is dissolved in water or biologically compatible aqueous buffer to obtain a solution of 20% w/v. PEI-poloxamer-188-PEI is dissolved in water or biological compatible aqueous buffer to obtain a solution of 25% w/v. Upon mixture of the two solutions, the hydrogel is formed within 2 seconds. If the mixture is performed on top of biological or prosthetic tissues or surfaces the hydrogel will strongly adhere to them.

### Example 4: Hydrogel synthesis with poloxamer 407

45% oxidized dextran is reacted with poloxamer 407 modified with ethylene diamine. Oxidized dextran is dissolved in water or biologically compatible aqueous buffer to obtain a solution of 10% w/v. NH2-poloxamer-407—NH2 is dissolved in water or biological compatible aqueous buffer to obtain a solution of 20% w/v. Upon mixture of the two solutions, the hydrogel is formed within 40 seconds. If the mixture is performed on top of biological or prosthetic tissues or surfaces the hydrogel will strongly adhere to them.

### Example 5: Thermal stability of hydrogels

The amine component of the hydrogel is based on the poloxamer which displays thermal gelation properties. While thermal gelation is not needed due to the fast and effective gelation achieved by the oxidized dextran crosslinked, once the material achieves physiological temperatures, the crosslinked poloxamer chains can still rearrange creating internal interactions that are able to dissipate energy enhancing the mechanical properties of the formed hydrogel.

Table 3 displays some formulations in which different poloxamers and CHO/NH2 ratios are used and their degradation after 24 h at 37° C. in presence of an excess of saline buffer.

TABLE 3
Thermal degradation of amine modified poloxamer/Oxidized
dextran formulations with different CHO/NH₂ ratios.

| | | | mmol - | Molar | | |
|---|---|---|---|---|---|---|
| | Volume | Mass | CHO or - | Ratio | Final | % |
| Formulation | (μL) | (mg) | NH₂) | CHO/NH₂ | concentration | Degraded |
| Poloxamer 407- | 100 | 20 | 0.0032 | 38.9 | 10.0% | 100% |
| (NH₂)₂ | | | | | | |
| Oxidized dextran | 100 | 20 | 0.1235 | | 10.00% | |
| Poloxamer 407- | 33 | 9.9 | 0.0016 | 32.6 | 4.9% | 91% |
| (NH₂)₂ | | | | | | |
| Oxidized dextran | 166 | 8.3 | 0.0512 | | 4.1% | |
| Poloxamer 407- | 100 | 30 | 0.0048 | 25.9 | 15.0% | 83% |
| (NH₂)₂ | | | | | | |
| Oxidized dextran | 100 | 20 | 0.1235 | | 10.0% | |
| Poloxamer 407- | 133 | 26.6 | 0.0042 | 19.3 | 13.3% | 70% |
| (NH₂)₂ | | | | | | |
| Oxidized dextran | 66 | 13.2 | 0.0815 | | 6.6% | |
| Poloxamer 407- | 133 | 22.61 | 0.0036 | 11.4 | 11.3% | 72% |
| (NH2)2 | | | | | | |
| Oxidized dextran | 66 | 6.6 | 0.0407 | | 3.3% | |
| Poloxamer 407- | 160 | 32 | 0.0051 | 9.7 | 16.0% | 40% |
| (NH₂)₂ | | | | | | |
| Oxidized dextran | 40 | 8 | 0.0494 | | 4.0% | |
| Poloxamer 407- | 133 | 26.6 | 0.0042 | 9.6 | 13.3% | 90% |
| (NH₂)₂ | | | | | | |
| Oxidized dextran | 66 | 6.6 | 0.0407 | | 3.3% | |
| Poloxamer 407- | 133 | 33.25 | 0.0053 | 7.7 | 16.7% | 10% |
| (NH₂)₂ | | | | | | |
| Oxidized dextran | 66 | 6.6 | 0.0407 | | 3.3% | |
| Poloxamer 407- | 133 | 22.61 | 0.0036 | 5.7 | 11.3% | 87% |
| (NH₂)₂ | | | | | | |
| Oxidized dextran | 66 | 3.3 | 0.0204 | | 1.6% | |
| Poloxamer 407- | 133 | 26.6 | 0.0042 | 4.8 | 13.3% | 75% |
| (NH₂)₂ | | | | | | |
| Oxidized dextran | 66 | 3.3 | 0.0204 | | 1.6% | |
| Poloxamer 188- | 100 | 25 | 0.0850 | 1.1 | 12.5% | 5% |
| (PEI)2 | | | | | | |
| Oxidized dextran | 100 | 15 | 0.0938 | | 7.5% | |

### Example 6: Mechanism of gelation

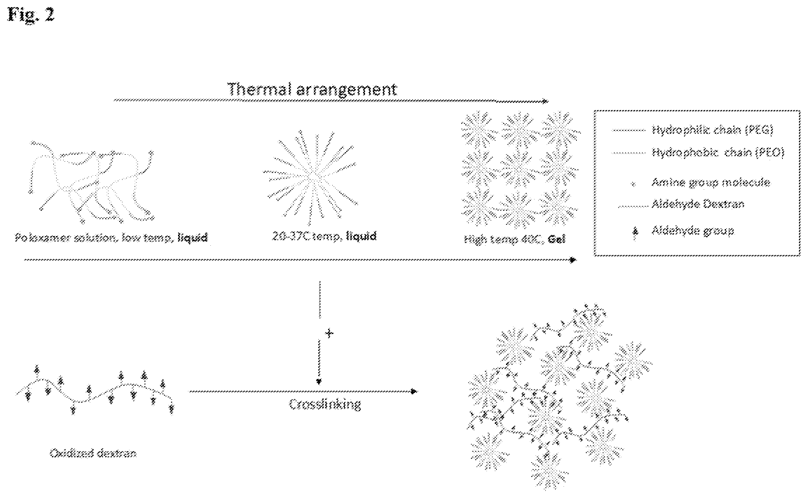

FIG. 2 displays the multiple interaction mechanisms of the composition. Amine modified poloxamer displays temperature responsive properties, presenting micelation at medium temperatures, and highly organized molecular arrangements at higher temperatures. In parallel, oxidized dextran can react with the terminal amines present in the poloxamer creating a strong, stable and temperature independent gelation.

### Example 7: Adhesion mechanisms of the adhesive material

The current invention is able to achieve strong and sustained adhesion to biological and prosthetic tissue by means of using multiple mechanisms of adhesion.

### Example 7A: Covalent adhesion mechanism

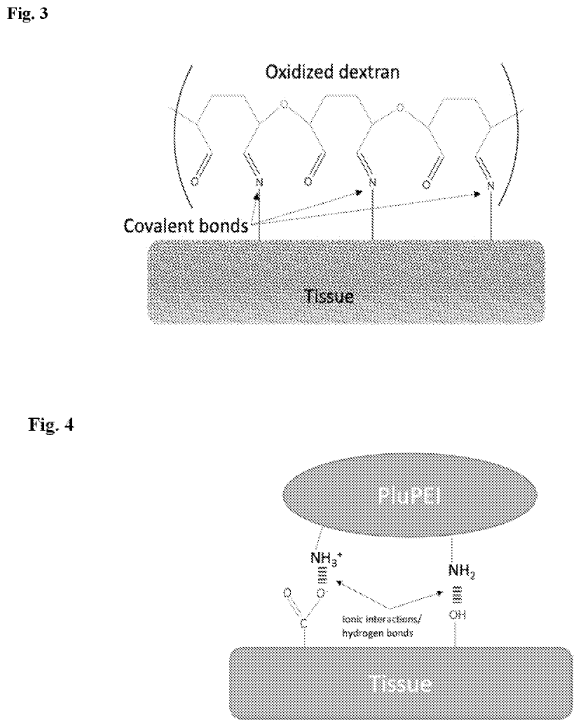

The oxidized dextran contains aldehyde groups that readily react with the amines present in the proteins and other molecules in the tissue surface. The covalent bond created between aldehyde and amines is an imine bond (FIG. 3). Imine bonds provide strong binding, but they are reversible overtime, allowing the material to detach and be eliminated as the tissue heals. This chemical reaction is fast but not instantaneous.

### Example 7B: Ionic adhesion mechanism

Ionic interactions gather a broad group of molecular interactions that provide an important contribution to the total adhesion. In this formulation, the amines present in the PluPEI molecule is the main contributor to the ionic interaction. Protonated amines (positively charged) can create interactions with negatively charged groups in the tissue surface such as carboxylic acids (FIG. 4). Amines can also create hydrogen bonds with functional groups in the tissue surface. Ionic interactions provide a very fast, “magnet-like, adhesion; however, alone, they are not strong enough to maintain material adhesion overtime.

### Example 7C: Mechanical interlocking adhesion mechanism

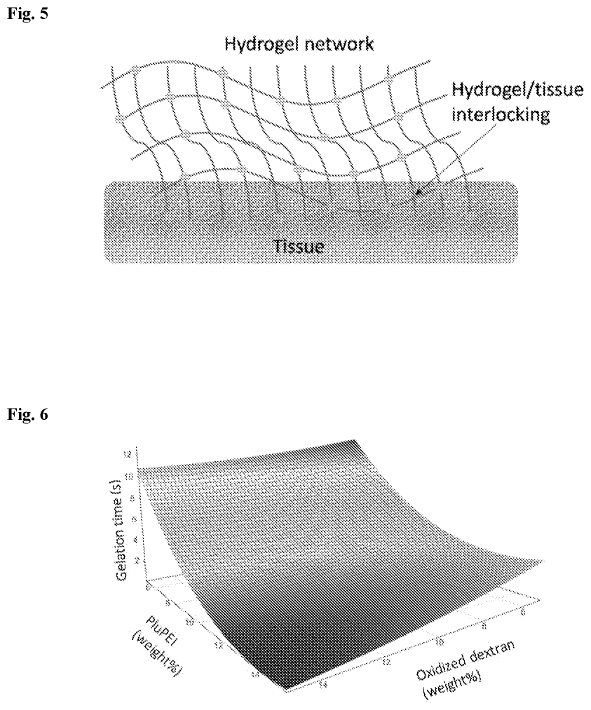

The tissue surface is like a porous matrix into which the sprayed liquid polymers can penetrate (at a molecular level). Once gelation occurs, the bulk of the spray will be interdigitated within the tissue creating an extra level of mechanical interaction (FIG. 5).

### Example 8: Gelation time of the adhesive material

Gelation time is an important parameter for certain applications. In certain embodiments, the adhesive material is able to achieve almost instant gelation without needing external triggers such as UV light or heat. This fast gelation enables the application in complex geometries accessed by minimally invasive techniques or endoscopy, where the adhesive formulation needs to target to very specific site without dripping (FIG. 6).

### Example 9: Swelling of the adhesive material

Swelling is an important property when delivering a hydrogel-based solution in a confined space. For gastrointestinal applications this is not a big concern if the hydrogel swells within an adequate range. Some commercial hydrogels can swell up to 1000% of their initial weight. However, some degree of swelling (≤200%) is desired as the hydrogel increase its thickness forms a better shielding to the underlaying wound. A higher percentage of water in the material is often associated with a higher degree of biocompatibility. Excessive hydrogel swelling is also associated with the loss of adhesion properties overtime.

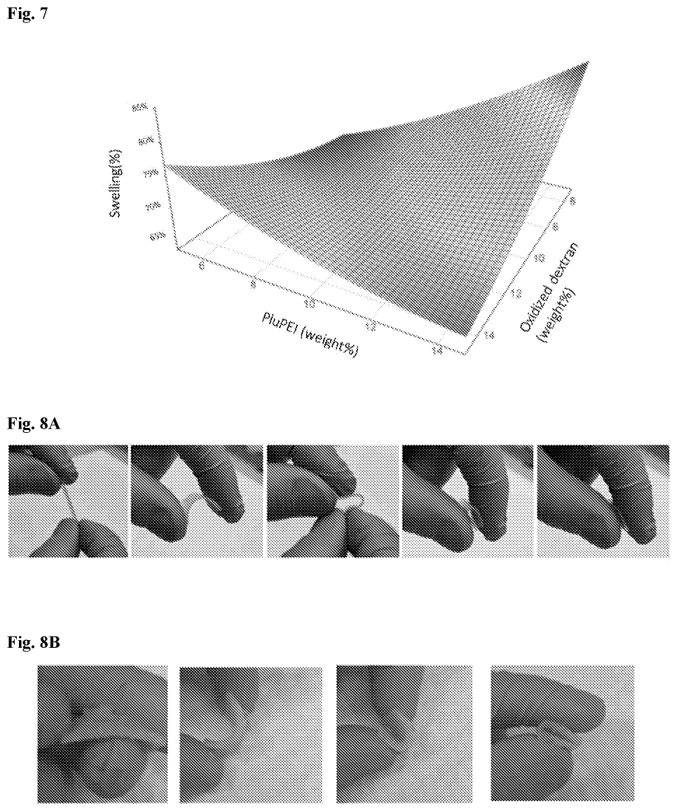

FIG. 7 displays the surface response analysis of hydrogels with a solid content ranging from 5% to 15% both for oxidized dextran and PluPEI. There is a clear ratio between the components that provides with minimum swelling. When both oxidized dextran and PluPEI have a similar solid content, the swelling values are constrained in the range of 65%-75%. Increasing the solid content beyond 14% will further decrease the swelling of the material if required. An excess of molecules from either of the components can deviate the polymeric network from its ideal structure and cause excessive swelling and even decreased stability.

### Example 10: Material flexibility of the adhesive material

Some sprayable materials become brittle or stiff after gelation. It is important that the mechanical properties of the hydrogel will remain similar to the properties of the underlaying intestinal tissue. Also, as there might be a variability in spraying volume from one clinician to the other, it is essential for the material to remain flexible even when a thick layer is applied.

In FIG. 8A a thin and in FIG. 8B a thick layer of the material are bent up to 180 degrees and the material does not create cracks or breaks. This test was performed 30 min after the material was sprayed to allow for its “complete” and potentially more stiff and brittle mechanical properties.

### Example 11: Burst pressure of the material in porcine colon tissue

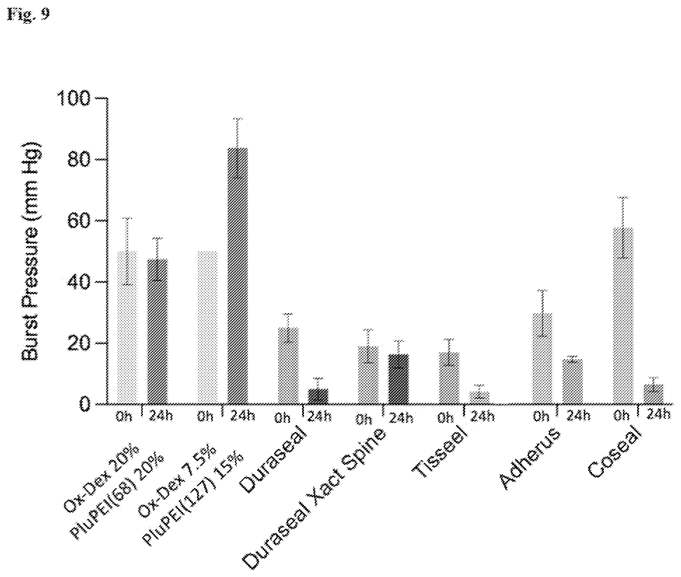

Most of the commercial materials lose their adhesion once they are incubated in physiological environments. For chronic protection and sealing, it is critical that the adhesion of the material is maintained overtime. In FIG. 9, in comparing several hydrogels including commercial materials, it was observed that in about 10 minutes after spraying, two formulations of oxidized dextran and PluPEI displayed superior burst pressures. Furthermore, materials comprising various ratios of oxidized dextran-PluPEI are formulations that either maintained or increased performance when tested 24 h after incubation in physiological environment.

### Example 12: Buffering capacity of the hydrogel

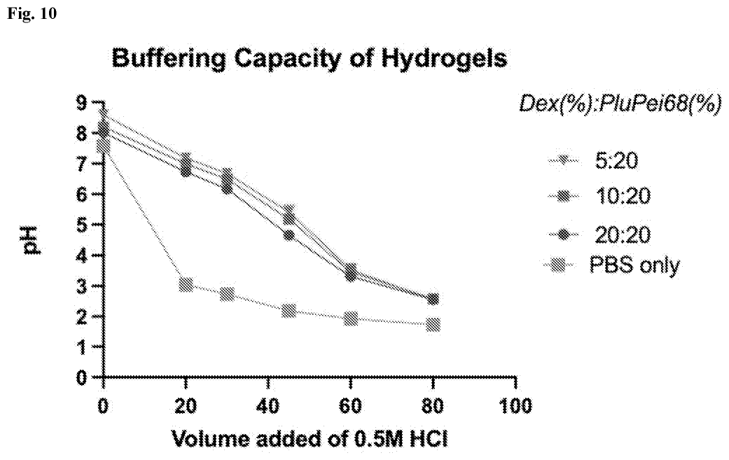

The high amine concentration found in the PluPEI component, provides the hydrogel with a strong buffering capacity. The capacity of neutralizing acid is of special interest in stomach applications in which the digestive secretions have a very low pH and may impede the wound healing process. In FIG. 10 several hydrogel formulations show a strong buffering capacity when adding 0.5 M HCl to a PBS solution containing 200 mg of hydrogel compared to PBS alone.

### Example 13: Hydrogel formation with pluronic poloxamer 338 and poloxamer 237

A material with a composition of 12% solid content of PluPEI (poloxamer 338) and 5% oxidized dextran forms a hydrogel in 10s and displays about 60% swelling after 24h in PBS. A material with the same concentration but with PluPEI (poloxamer 237) forms a hydrogel in 5 seconds and displays about 45% swelling after 24h in PBS.

### Example 14: Swelling of specific formulations

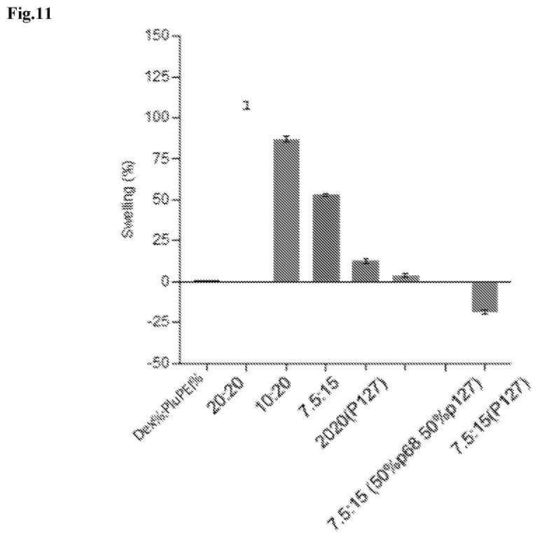

There are multiple factors that influence the swelling of the resulting hydrogels. By modifying the type of poloxamer used in the core of the PluPEI, the ratio between components and the % of solid content, a broad range of swelling values can be obtained (FIG. 11).

### Example 15: Drug release from the hydrogel

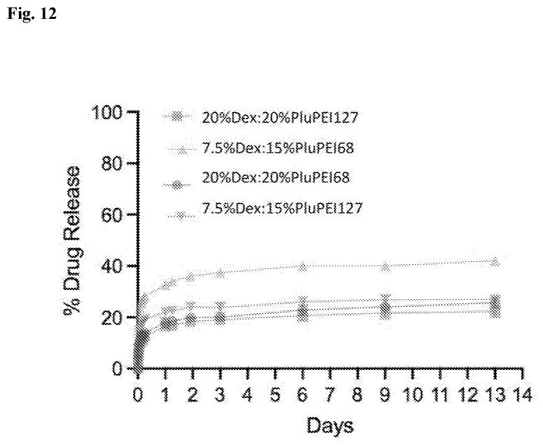

The high biocompatibility profile and sustain adhesion over time makes the oxidized dextran PluPEI hydrogel an exceptional depot for drug release. FIG. 12 displays the in-vitro drug release profile in PBS of four formulations loaded with 2 mg/mL of acriflavine.

### Example 16: Low solid content formulations

Hydrogels can be obtained with very low solid concentration for those applications that require less demanding mechanical properties or a faster bioelimination. One low solid content hydrogel is formed by the combination of 6% PluPEI (407) and 2.5% 0xDex. This hydrogel has a final total solid content of 4.25% and displays a stability of 7 days.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

We claim:

1. A biocompatible adhesive material, comprising:
a block copolymer comprising one or more polyethylene oxide (PEO) blocks, one or more polypropylene oxide (PPO) blocks, and at least one primary amine group; and
an oxidized polysaccharide comprising at least two aldehyde moieties;
wherein the adhesive material is crosslinked by a plurality of imine moieties formed from the at least one primary amine of the block copolymer and the at least two aldehyde moieties of the oxidized polysaccharide; and the adhesive material is a hydrogel.

2. The biocompatible adhesive material of claim 1, wherein the block copolymer has a molecular weight of about 1,000 Da to about 25,000 Da, or about 1,000 Da to about 20,000 Da.

3. The biocompatible adhesive material of claim 1 or 2, wherein the block copolymer is inverse thermosensitive.

4. The biocompatible adhesive material of any one of claims 1-3, wherein the block copolymer comprises 1 to about 50 primary amines.

5. The biocompatible adhesive material of any one of claims 1-4, wherein the block copolymer comprises 1 to about 25 primary amines.

6. The biocompatible adhesive material of any one of claims 1-5, wherein the block copolymer has a PEO blocks: PPO blocks ratio of about 50:50 to about 80:20.

7. The biocompatible adhesive material of any one of claims 1-6, wherein the block copolymer is FG-(poloxamer 188)-FG, FG-(poloxamer 237)-FG, FG-(poloxamer 338)-FG, FG-(poloxamer 407)-FG, or a combination of any of them, wherein FG independently for each occurrence represents H or a primary amine group, at least one occurrence of FG is not H.

8. The biocompatible adhesive material of any one of claims 1-7, wherein independently for each occurrence the primary amine group is 1,2-diaminoethane, tris (2-aminoethyl) amine, or a low molecular weight (e.g., less than about 2,000, 1,000, 500, or 250 Daltons) polyethyleneimine.

9. A biocompatible adhesive material, comprising:
a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine; and
an oxidized polysaccharide comprising at least two aldehyde moieties;
wherein the adhesive material is crosslinked by a plurality of imine moieties formed from the at least one primary amine of the linker-amine moiety of the functionalized poloxamer and an aldehyde moiety of the oxidized polysaccharide; and
the adhesive material is a hydrogel.

10. The biocompatible adhesive material of claim 9, wherein both occurrences of FG represent a linker-amine moiety.

11. The biocompatible adhesive material of claim 9 or 10, wherein the functionalized poloxamer has a molecular weight of about 1,000 Da to about 25,000 Da, or about 1,000 Da to about 20,000 Da.

12. The biocompatible adhesive material of any one of claims 9-11, wherein the functionalized poloxamer is inverse thermosensitive.

13. The biocompatible adhesive material of any one of claims 9-12, wherein each linker-amine moiety comprises 1 to about 50 primary amines.

14. The biocompatible adhesive material of any one of claims 9-13, wherein each linker-amine moiety comprises 1 to about 25 primary amines.

15. The biocompatible adhesive material of any one of claims 9-14, wherein the functionalized poloxamer has a PEO: PPO ratio of about 50:50 to about 80:20.

16. The biocompatible adhesive material of any one of claims 9-15, wherein the functionalized poloxamer is FG-(poloxamer 188)-FG, FG-(poloxamer 237)-FG, FG-(poloxamer 338)-FG, FG-(poloxamer 407)-FG, or a combination of any of them.

17. The biocompatible adhesive material of any one of claims 9-16, wherein independently for each occurrence the amine moiety of the linker-amine moiety is 1,2-diaminoethane, tris (2-aminoethyl) amine, or a low molecular weight (e.g., less than about 2,000, 1,000, 500, or 250 Daltons) polyethyleneimine.

18. The biocompatible adhesive material of any one of claims 1-17, wherein the oxidized polysaccharide has a molecular weight of about 1,000 Da to about 100,000 Da.

19. The biocompatible adhesive material of any one of claims 1-18, wherein the degree of functionalization of the oxidized polysaccharide is about 10% to about 75%.

20. The biocompatible adhesive material of any one of claims 1-19, wherein the oxidized polysaccharide comprises 2 to 100 aldehyde moieties.

21. The biocompatible adhesive material of claim 20, wherein the oxidized polysaccharide comprises 40 to 80 aldehyde moieties.

22. The biocompatible adhesive material of claim 21, wherein the oxidized polysaccharide comprises 50 to 70 aldehyde moieties.

23. The biocompatible adhesive material of any one of claims 1-22, wherein the oxidized polysaccharide is a dextran.

24. The biocompatible adhesive material of claim 23, wherein the dextran has a molecular weight of about 10 kDa.

25. The biocompatible adhesive material of claim 23 or 24, wherein about 50% of the hydroxyl groups of the dextran are oxidized to aldehydes.

26. The biocompatible adhesive material of any one of claims 1-25, wherein the adhesive material has a flexibility to bend up to 180 degrees.

27. A method for treating, adhering, or sealing one or more biological tissues or prosthetic materials, the method comprising:
providing a block copolymer comprising one or more polyethylene oxide (PEO) blocks, one or more polypropylene oxide (PPO) blocks, and at least one primary amine group;
providing a second component comprising an oxidized polysaccharide comprising at least two aldehyde moieties;
combining the first component and the second component, thereby producing an adhesive formulation;
contacting the adhesive formulation with a surface of one or more biological tissues; and
allowing the adhesive formulation to cure in contact with the surface of the one or more biological tissues.

28. The method of claim 27, further comprising the step of adjusting the concentrations of the first component or the second component to compensate for the characteristics of the biological tissue.

29. The method of claim 27 or 28, wherein the block copolymer has a molecular weight of about 1,000 Da to about 25,000 Da, or about 1,000 Da to about 20,000 Da.

30. The method of any one of claims 27-29, wherein the block copolymer is inverse thermosensitive.

31. The method of any one of claims 27-29, wherein the block copolymer comprises 1 to about 50 primary amines.

32. The method of any one of claims 27-29, wherein the block copolymer comprises 1 to about 25 primary amines.

33. The method of any one of claims 27-32, wherein the block copolymer has a PEO blocks: PPO blocks ratio of about 50:50 to about 80:20.

34. The method any one of claims 27-33, wherein the block copolymer is FG-(poloxamer 188)-FG, FG-(poloxamer 237)-FG, FG-(poloxamer 338)-FG, FG-(poloxamer 407)-FG, or a combination of any of them, wherein FG independently for each occurrence represents H or a primary amine group, at least one occurrence of FG is not H.

35. The method of any one of claims 27-34, wherein independently for each occurrence the primary amine group is 1,2-diaminoethane, tris (2-aminoethyl) amine, or a low molecular weight (e.g., less than about 2,000, 1,000, 500, or 250 Daltons) polyethyleneimine.

36. A method for treating, adhering, or sealing one or more biological tissues or prosthetic materials, the method comprising:
providing a first component comprising a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine;
providing a second component comprising an oxidized polysaccharide comprising at least two aldehyde moieties;
combining the first component and the second component, thereby producing an adhesive formulation;
contacting the adhesive formulation with a surface of one or more biological tissues; and
allowing the adhesive formulation to cure in contact with the surface of the one or more biological tissues.

37. The method of claim 36, further comprising the step of adjusting the concentrations of the first component or the second component to compensate for the characteristics of the biological tissue.

38. The method of claim 36, wherein the functionalized poloxamer has a molecular weight of about 1,000 Da to about 25,000 Da, or about 1,000 to about 20,000 Da.

39. The method of any one of claims 36 to 38, wherein the functionalized poloxamer is inverse thermosensitive.

40. The method of any one of claims 36 to 39, wherein each linker-amine moiety comprises 1 to about 50 primary amines.

41. The method of any one of claims 36 to 40, wherein each linker-amine moiety comprises 1 to about 25 primary amines.

42. The method of any one of claims 36 to 41, wherein the functionalized poloxamer has a PEO: PPO ratio of about 50:50 to about 80:20.

43. The method of any one of claims 36 to 42, wherein the functionalized poloxamer is FG-(poloxamer 188)-FG, FG-(poloxamer 237)-FG, FG-(poloxamer 338)-FG, FG-(poloxamer 407)-FG, or a combination of any of them.

44. The method of any one of claims 35 to 43, wherein independently for each occurrence the amine moiety of the linker-amine moiety is 1,2-diaminoethane, tris (2-aminoethyl) amine, or a low molecular weight (e.g., less than about 2,000, 1,000, 500, or 250 Daltons) polyethyleneimine.

45. The method of any one of claims 27 to 44, wherein the oxidized polysaccharide has a molecular weight of about 1,000 Da to about 100,000 Da.

46. The method of any one of claims 27 to 45, wherein the degree of functionalization of the oxidized polysaccharide is about 10% to about 75%.

47. The method of any one of claims 27 to 46, wherein the oxidized polysaccharide comprises 2 to 100 aldehyde moieties.

48. The method of claim 47, wherein the oxidized polysaccharide comprises 40 to 80 aldehyde moieties.

49. The method of claim 48, wherein the oxidized polysaccharide comprises 50 to 70 aldehyde moieties.

50. The method of any one of claims 27 to 49, wherein the oxidized polysaccharide is a dextran.

51. The method of claim 50, wherein the dextran has a molecular weight of about 10 kDa.

52. The method of claim 50 or 51, wherein about 50% of the hydroxyl groups of the dextran are oxidized to aldehydes.

53. The method of any one of claims 27 to 52, wherein curing of the adhesive formulation in contact with the one or more biological tissues comprises covalent bonding, ionic bonding, mechanical interlocking, or a combination thereof.

54. The method of any one of claims 27 to 37, wherein the adhesive formulation has a gelation time of about 1 second to about 100 seconds.

55. The method of claim 54, wherein the adhesive formulation has a gelation time of about 40 seconds to about 80 seconds.

56. The method of any one of claims 27 to 55, wherein the adhesive formulation has a swelling value of about 30% to about 90%.

57. The method of claim 56, wherein the adhesive formulation has a swelling value of about 40% to about 75%.

58. The method of any one of claims 27 to 57, wherein the first component and the second component are combined on the surface of the one or more biological tissues; and the adhesive formulation is formed on the surface of the one or more biological tissues;
optionally wherein the adhesive formulation is formed on a suture line associated with a PTFE or polyester vascular graft.

59. The method of any one of claims 27 to 58, wherein the one or more biological tissues comprise human tissue.

60. The method of any one of claims 27 to 59, wherein the first component, second component, or both further comprise an additive selected from the group consisting of foaming agents, pH modifiers, thickeners, antimicrobial agents, colorants, surfactants, and radio-opaque agents.

61. The method of any one of claims 27 to 60, wherein the first component is an aqueous solution.

62. The method of any one of claims 27 to 61, wherein the second component is an aqueous solution.

63. The method of any one of claims 27 to 62, wherein the first component, second component, or both components, comprise an effective amount of at least one drug.

64. The method of claim 63, further comprising the step of permitting the at least one drug to diffuse from the adhesive formulation into the biological tissue.

65. The method of any one of claims 36 to 64, wherein the adhesive formulation reduces inflammation, enhances healing, or both in the biological tissue.

66. The method of any one of claims 36-65, wherein the biological tissues is a gastrointestinal (GI) tissue.

67. The method of any one of claims 36-66, wherein the first component and the second component are provided using an endoscopic technique.

68. A kit, comprising:
a first component, comprising a block copolymer comprising one or more polyethylene oxide (PEO) blocks, one or more polypropylene oxide (PPO) blocks, and at least one primary amine group; and
a second component, comprising an oxidized polysaccharide comprising at least two aldehyde moieties;
a first syringe; and
instructions for use.

69. A kit, comprising:
a first component, comprising a functionalized poloxamer represented by FG-PEO—PPO-PEO-FG, wherein FG independently for each occurrence represents H or a linker-amine moiety, at least one occurrence of FG is not H, and independently for each occurrence the linker-amine moiety comprises at least one primary amine; and
a second component, comprising an oxidized polysaccharide comprising at least two aldehyde moieties;
a first syringe; and
instructions for use.

70. The kit of claim 68 or 69, wherein the first syringe comprises a first reservoir and a second reservoir; the first reservoir comprises the first component; and the second reservoir comprises the second component; optionally the first syringe comprises a mixing tip or a spray nozzle.

71. The kit of claim 70, further comprising a second syringe.

72. The kit of claim 71, wherein the first component is stored in the first syringe, and the second component is stored in the second syringe.

73. The kit of any one of claims 68-72, wherein each of the first syringe and the second syringe comprise a mixing tip or a spray nozzle.

74. The kit of any one of claims 68-73, wherein the instructions explain how to select an appropriate concentration of the first component or the second component or both to compensate for the characteristics of one or more biological tissues.

75. The kit of any one of claims 68-74, wherein the first component or the second component or both comprise a drug.

76. A drug delivery composition, comprising:
a biocompatible adhesive material of any one of claims 1-26; and
an effective amount of at least one drug;
wherein the at least one drug is admixed with the biocompatible adhesive material.

77. The drug delivery composition of claim 76, wherein the composition is in contact with a biological tissue.
